(19) United States
(12) Patent Application Publication
(10) Pub. No.: US 2012/0295814 A1
(43) Pub. Date: Nov. 22, 2012

(54) CA-125 Immune Complexes as Biomarkers of Ovarian Cancer

(75) Inventors: Daniel Cramer, Chestnut Hill, MA (US); Brian Liu, Somerville, MA (US); Dennis O'Rourke, West Townsend, MA (US)

(73) Assignee: THE BRIGHAM AND WOMEN'S HOSPITAL, INC., Boston, MA (US)

(21) Appl. No.: 13/520,665

(22) PCT Filed: Jan. 7, 2011
(86) PCT No.: PCT/US11/20465

§ 371 Date: Jul. 5, 2012

Related U.S. Application Data
(60) Provisional application No. 61/293,551, filed on Jan. 8, 2010.

(51) Int. Cl. G01N 33/574; G01N 33/577; C40B 30/04

(52) U.S. Cl. 506/9; 436/501

(57) ABSTRACT

The invention is directed to assays of CA-125 immune complexes that can be used diagnostically. It also includes glass or plastic plates or slides on which the monoclonal antibodies against CA-125 have been immobilized and kits containing these plates or slides.

### CROSS REFERENCE TO RELATED APPLICATIONS

The present application claims priority to, and the benefit of, U.S. provisional application 61/293,551, filed on Jan. 8, 2010. This prior application is hereby incorporated by reference in its entirety.

### STATEMENT OF GOVERNMENT SUPPORT

This invention was made with Government support under Grant Nos. DK078566, CA105009, CA86381 awarded by the National Institutes of Health. The United States Government therefore has certain rights in the invention.

### FIELD OF THE INVENTION

The present invention is directed to assays for measuring CA-125 immune complexes in a biological fluid. These assays can be coupled with assays of free CA-125 levels to diagnostically evaluate a woman for ovarian cancer. The invention also includes solid supports with immobilized monoclonal antibodies to CA-125 that can be used in the detection of CA-125 or included as part of a kit containing components needed for diagnostic assays of ovarian cancer.

### BACKGROUND OF THE INVENTION

Ovarian cancer is the fifth leading cause of death from cancer in US women. In about 70% of cases, the diagnosis is not made until the cancer is in an advanced state, at a time when the five-year survival rate for patients is only about 28% (Ries, et al., SEER Cancer Stat. Rev. 1973-1995 (1998)). Such cases are typically treated by both surgery and chemotherapy and repeat treatment is required in most patients when the disease recurs. Often, the first indication of a recurrence is an elevation of CA-125. In contrast to the poor prognosis for patients diagnosed as having advanced ovarian cancer, the five year survival rate for women found to have localized disease is about 95% and surgery alone is often curative in such cases. These observations provide a rationale for the development of tests that are effective at identifying ovarian cancer at an early stage and which are better at monitoring patients for disease recurrence.

Thus far, no markers have been formally approved for screening for ovarian cancer and only two have been approved in the United States for monitoring disease recurrence, CA-125 and HE4. Although elevated blood levels of CA-125 usually correlate with the presence of cancer, there is no apparent elevation in about 20-30% of patients that have ovarian cancer at the time examined. While some of these cases may occur simply because an ovarian tumor does not express CA-125 (e.g. most mucinous type ovarian cancers), another explanation is that CA-125 antigen binds to anti-CA-125 antibodies in a patient's blood to form complexes that prevent an accurate determination of the total amount of CA-125 present.

### SUMMARY OF THE INVENTION

In its first aspect, the invention is directed to a method of determining the amount of CA-125 immune complex in a test biological sample (preferably blood, plasma, or serum) derived from a woman by contacting the sample with an antibody that binds to human anti-CA-125 antibody. Normally, this would be done by first separating the CA-125 immune complexes from free antibody, e.g., based on size or by using an immobilized antibody that binds to CA-125. The human anti-CA-125 antibody detected in assays (typically a human IgG) is derived from the bloodstream of the woman donating the biological sample and forms the immune complexes by binding to CA-125 released into her blood from ovarian cells. Among women that may be selected for testing are women with clinical factors indicating an increased risk of having or developing ovarian cancer, women previously diagnosed as having ovarian cancer and being tested for disease recurrence and women who have had a diagnostic assay performed measuring primarily free CA-125 levels in a sample of blood serum or plasma during the previous three months. The term “free CA-125” and “free antibody” as used herein refer to proteins that are not in an immune complex. The term “immune complex” refers to a complex of compounds that includes, at a minimum, CA-125 and a human antibody bound to the CA-125.

In a preferred aspect, immune complexes are assayed by obtaining a biological sample from a woman and then incubating this sample with a monoclonal antibody to CA-125 that has been immobilized on a solid support. The incubation is carried out under conditions permitting the binding of the monoclonal antibody to CA-125, e.g. at 35-40° C. for a period of 0.25-2.0 hours. At the end of the incubation, the solid support is separated from the test biological sample, i.e., the solid support is removed and, typically, washed, and a second incubation is carried out with a detectably labeled antibody that binds to human antibody. When this is completed, the solid support is separated from unbound antibody and the amount of detectable label bound to the solid support is quantitated.

More generally, the invention encompasses diagnostically testing a woman for ovarian cancer, by determining the amount of CA-125 immune complexes (or CA-125 present in immune complexes) in a biological sample obtained from the woman. Although an appropriate biological sample may be taken from any woman, women with a family history of ovarian cancer or other clinical factors suggesting an elevated risk for this disease are of particular interest. In an especially preferred embodiment, the tests will be used in detecting possible recurrences in women that have already been diagnosed as having ovarian cancer and who have received treatment. Assays of complexes may allow the detection of recurrences in these women in instances where they appear to have normal levels of CA-125 using other tests.

It is expected that assays for immune complexes (or CA-125 that is in immune complexes) will often be used together with assays for free CA-125. Both the assays for free protein and complexed protein should be performed on the same type of biological sample (preferably blood, plasma or serum) and, ideally, the exact same sample would be divided into aliquots and used in each assay. In preferred embodiments: a) the biological sample is blood, plasma, or serum; b) the solid support is a plate or slide made of glass, plastic or metal; c) the antibody used to detect human antibody in assays of complexed CA-125 is directed at a human IgG antibody; and the detectable label is a fluorescent label, most preferably Cy3 or Cy5.

A preferred assay for free CA-125 involves incubating the test biological sample with a monoclonal antibody to CA-125 that has been immobilized on a solid support, i.e., with the same type of support described above in connection with the detection of immune complexes of CA-125. The incubation should be carried out under conditions permitting the binding of the monoclonal antibody to CA-125, e.g., the conditions described above and, at the end of the incubation, the solid support with immobilized monoclonal antibody and bound immune complexes is separated from the test biological sample. A second incubation is then performed in which the solid support with immobilized monoclonal antibody and bound CA-125 is contacted with a second antibody (i.e., other than the monoclonal antibody bound to the solid support) that binds to CA-125. At the end of the second incubation, the amount of bound second antibody determined. In one embodiment, this is done directly by using a second antibody that has been detectably labeled. In an alternative embodiment, the second antibody is not labeled and the solid support with bound second antibody is separated from unbound second antibody. A third incubation is then performed with a detectably labeled third antibody that binds to the second antibody. After the third incubation, the solid support with bound detectably labeled antibody is separated from unbound antibody and the amount of detectable label bound to the solid support is quantitated.

In all of the assays described herein, it will be recognized that standard methods for carrying out incubations and for washing plates between incubations may be included as needed and standard variations in procedures will be readily apparent to those of skill in the art. As the result of assays, the amount of free CA-25 may be determined and added to the amount of immune complex or CA-125 in immune complexes. This, “total CA-125” level may be compared with CA-125 levels in one or more control samples, e.g., derived from the general population or from women known to be free of ovarian cancer. When the total amount of CA-125 in the test sample is higher than in the control samples, this is an indication that the woman from which the test sample was derived has ovarian cancer and should be further examined.

In another aspect, the invention is directed to solid supports (preferably a plate or slide made of glass, plastic or metal) that have been used to immobilize monoclonal antibody against CA-125, preferably at discrete locations (e.g., in the wells of a multiwell plate) and at more than one concentration. The solid supports may be part of kit that also includes other components used in the assays described herein. These other components may include labeled or unlabeled antibody against CA-125 (apart from antibody immobilized on a solid support), labeled or unlabeled antibody recognizing human antibody (preferably IgG) and/or one or more control samples.

The invention also encompasses assays for detecting free anti-CA-125 antibody in a biological sample of blood, serum or plasma. Although any method for detecting such antibody can be used, a preferred method is described in US 2009/0075305 and US 2008/0081339, both of which are incorporated by reference herein in their entirety (see also Qin, et al., Proteomics 6:3199-209 (2006)). In brief, this assay would involve: a) immobilizing a monoclonal antibody to CA-125 on a solid support; b) saturating the immobilized antibody with CA-125 to form an assay-ready plate; c) incubating the biological sample with the assay-ready plate under conditions permitting the binding of antibody in the sample to the CA-125 that has been bound to the plate by the immobilized antibody; d) detecting the anti-CA-125 antibody using a second antibody that is attached to a detectable label. Routine mixing and washing steps can be incorporated into the assay as needed and many simple variations are possible that will be readily apparent to those of skill in the art. The assay for CA-125 antibody can be used either alone or in conjunction with the assays for free and immune complexed CA-125 described above.

### BRIEF DESCRIPTION OF THE DRAWINGS

FIG. 1 is a schematic diagram showing the way in which assays may be conducted for the purpose of detecting serum levels of free CA-125 and CA-125 that has been complexed with human antibody. Also shown are the results of assays performed on samples from ovarian cancer patients. Results suggest that patients with high free CA-125 levels generally have low levels of complexed antigen and vice versa. This suggests that the absence of elevated CA-125 levels in some ovarian cancer patients may be due to the use of assays that only, or at least preferentially, detect free antigen.

FIG. 2 depicts an assay that may be used to detect free CA-125 in a subject. The natural conformation of the CA-125 is maintained by using an immobilized monoclonal antibody (MUC16) to bind antigen to plates. After the binding of the CA-125 from serum, plates are incubated with rabbit anti-CA-125 antibody and with labeled goat anti rabbit antibody.

FIG. 3 is a diagram showing the way in which rabbit anti-CA-125 may fail detect CA-125. In the figure, rabbit anti-CA-125 antibody is blocked from binding to antigenic sites on the CA-125 protein by bound human antibody.

### DETAILED DESCRIPTION OF THE INVENTION

The present invention is based upon the development of an assay for the identification of CA-125 immune complexes. The assay can be used in combination with an assay of free CA-125 to assess total CA-125 levels in a sample. CA-125 levels determined in this manner provide a way of identifying women that have ovarian cancer.

Assay of Free CA-125

One method of carrying out assays of free CA-125 is based upon an adaptation of procedures described in US 2008/0081339 and US 2009/0075305 (incorporated herein by reference in their entirety). The first step in the procedure involves immobilizing monoclonal antibodies that bind to CA-125 on a solid support. Antibodies of this type may be purchased commercially or, alternatively, can be developed using methods well known in the art. If desired, fragments derived from the monoclonal antibodies that maintain the ability to specifically recognize CA-125 antigen may also be used. Methods for attaching proteins to support such as slides or plates are well known in the art (see generally, Rusmini, et al., Biomacromolecules 8:1775-1789 (2007); Wilson, et al., Curr. Opin. Chem. Biol. 6:81-85 (2002); Kalia, et al., Biocunjugate Chem. 18:1064-1060 (2007); Zhu, et al., Curr. Opin. Chem. Biol. 7:55-63 (2003)).

Once the array of immobilized CA-125 antibodies has been prepared, the next step is to prepare the biological samples that will undergo testing. One or more test samples of serum, plasma or blood are removed from a test subject, typically a woman who is being screened for the presence of ovarian cancer or for recurrence of ovarian cancer. Women may also be selected for testing because they are believed to be at a high risk for ovarian cancer on the basis of a family history of the disease or because they carry a predisposing mutation of the BRCA1 and BRCA2 gene. Of particular interest for testing are women with a history of non-mucinous ovarian cancer who did not have an elevated CA-125 level at presentation. One or more “control” samples of blood, plasma or serum derived, for example, from individuals that are known not to not have the disease (or be at familial risk for it) may also be prepared and included in assays to serve as a basis for comparison. Alternatively, comparisons can be made between test sample results and results based upon prior assays.

The CA-125 in the test and control samples may be partially purified prior to assay and/or diluted in an incubation buffer, although these steps are not essential. The incubation buffer may, for example, consist of any type of standard buffer used in handling proteins, e.g., PBS. A chosen amount of the samples is incubated with the array of immobilized monoclonal antibodies, e.g., at about room temperature for a period ranging from 15 minutes to 2 hours. At the end of this time, unbound material is removed and the solid support is washed and then incubated with anti-CA-125 antibodies, e.g., derived from rabbits. These antibodies may or may not be detectably labeled, i.e., treated with a substance that permits detection and quantitation such as a fluorescent or radioactive label.

If the anti-CA-125 antibodies have been labeled, then the amount of label bound to the slide or plate can be determined and should reflect the amount of free CA-125 in the sample. If the antibody against CA-125 has not been detectably labeled, the solid support or plates are incubated again, this time with an antibody that recognizes the anti-CA-125 antibody and that is detectably labeled. For example, if rabbit anti-CA-125 antibody was used in the prior incubation, then goat anti-rabbit Cy5-labeled antibodies may be used next and the final support scanned for label.

Assay of CA-125 Immune Complexes

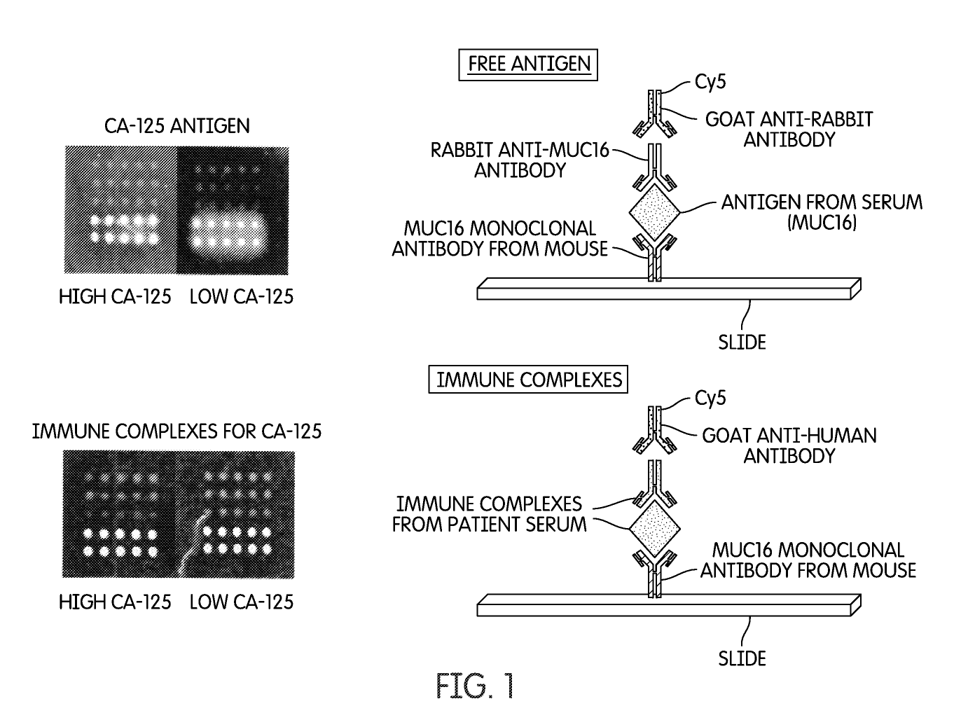

Assays for immune complexes containing CA-125 bound to antibody are performed in a manner similar to the assays described above for free CA-125. However, after the incubation of sample with anti-CA-125 monoclonal antibody immobilized on a solid support, the bound complex is incubated with detectably labeled anti-human antibody. The difference between the assay for free CA-125 and complexed CA-125 can be seen in FIG. 1. The lower diagram in this figure illustrates the detection of bound complex using fluorescently labeled goat antibody that binds to human antibody (e.g., human IgG). After these antibodies bind, the amount of fluorescence associated with the solid support can be determined, e.g., in a scanner that measures fluorescence.

Alternative Assays

The most essential feature of the assays described above is the use of an antibody that detects the presence of human antibody bound to CA-125 in complexes. Other aspects of the assays are preferred but not essential.

Use of Assays

In general, assays for free and complexed CA-125 should be used together to determine the total amount of CA-125 present in a test sample. The total determined is then compared with the levels of CA-125 in individuals known to be free of ovarian cancer, levels in the general population or levels in some other appropriate control group. If the amount of total CA-125 is elevated in the test sample relative to the controls, this is an indication that the subject from which the sample was obtained has ovarian cancer and further examination of the subject should be carried out to confirm that this is the case.

The assays of free and immune complexed CA-125 may be performed in any order, i.e., the assay of free CA-125 may be performed before or after the assay of the complexed protein. Alternatively, the assay of the complexed CA-125 may be limited to patients where levels of free CA-125 are only marginally elevated or there are other factors that lead to uncertainty regarding the reliability of the free CA-125 assay. The assays may be performed as part of a screening procedure for the general population, in patients suspected of having ovarian cancer or in ovarian cancer patients that have already been diagnosed. In the latter case, the assays may be used to help in monitoring disease progression or the effectiveness of therapies.

Finally, the measurement of free and complexed CA-125 can be combined into a single assay by first capturing free and complexed CA-125 on a solid support with immobilized antibodies and then either simultaneously or sequentially carrying out incubations with a first labeled antibody that binds to CA-125 and with a second labeled antibody that binds to human antibody. Preferably the labels used on the first and second labeled antibodies will be distinguishable from one another. For example, one antibody may be labeled with Cy3 and the other with Cy5. Simple variations on this procedure will be readily apparent to those of skill in the art.

Kits

Solid supports, e.g., microarray plates or slides, containing bound monoclonal antibody against CA-125 may be sold or included as part of a kit that contains other components needed to perform the assays described herein. For example, the kit may additionally include: labeled or unlabeled unbound antibody against CA-125; labeled or unlabeled antibody recognizing human antibody; control samples; buffers or other components. In addition, kits will typically also include instructions describing how the various components can be used in performing diagnostic assays for ovarian cancer.

### EXAMPLES

The present example describes a protein microarray method used to measure CA-125 antigen and CA-125 present in immune complexes in ovarian cancer patients.

Methods A protein capture microarray platform is arrayed with mouse monoclonal antibodies to CA-125 spotted onto a nano-particle chip and directed towards native purified human CA-125. Serum from cases and controls is added to the slides in respective wells so that the free antigens may be detected. The slides are then washed and incubated with rabbit anti-CA-125 antibodies. After another washing, the slides are incubated with goat anti-rabbit Cy5-labeled antibodies. The slides are then scanned for fluorescence levels and quantified using ScanArray.

For the portion of the experiment focusing on immune complexes, we begin with the slides coated with monoclonal antibodies to CA-125. Patient serum is then added. The printed antibodies will capture the CA-125 and any associated immune complexes such as antigen-autoantibody complexes. After washing, the slides are followed with Cy5-tagged goat anti-human antibodies. The slides are then scanned for fluorescence levels and quantified using ScanArray. FIG. 1 illustrates each of the methods.

Results

Ten cases with advanced serous ovarian cancer which presented with the expected high CA-125 levels were matched by age to ten advanced serous cases which had CA-125<100 U/ml. FIG. 1 shows the fluorescence of CA-125 antigen and corresponding immune complexes. In 8 out of 10 pairs, the cases with high levels of CA-125 appeared to have a lower concentration of CA-125 immune complexes than the cases with lower levels of CA-125. For each array, we spotted the monoclonal antibodies to CA-125 in various dilutions (400 ug, 200 ug, 100 ug), with each dilution spotted 5× to demonstrate reproducibility. Cy3-conjugated BSA was used as an internal control as well as for a ratio-based normalization of fluorescence levels. The corresponding table, table 1, demonstrates that there is approximately a 100% increase in fluorescence levels of immune complexes for CA-125 in the case with a low CA-125 free antigen level when compared to the case with a high CA-125 free antigen level.

### CONCLUSION

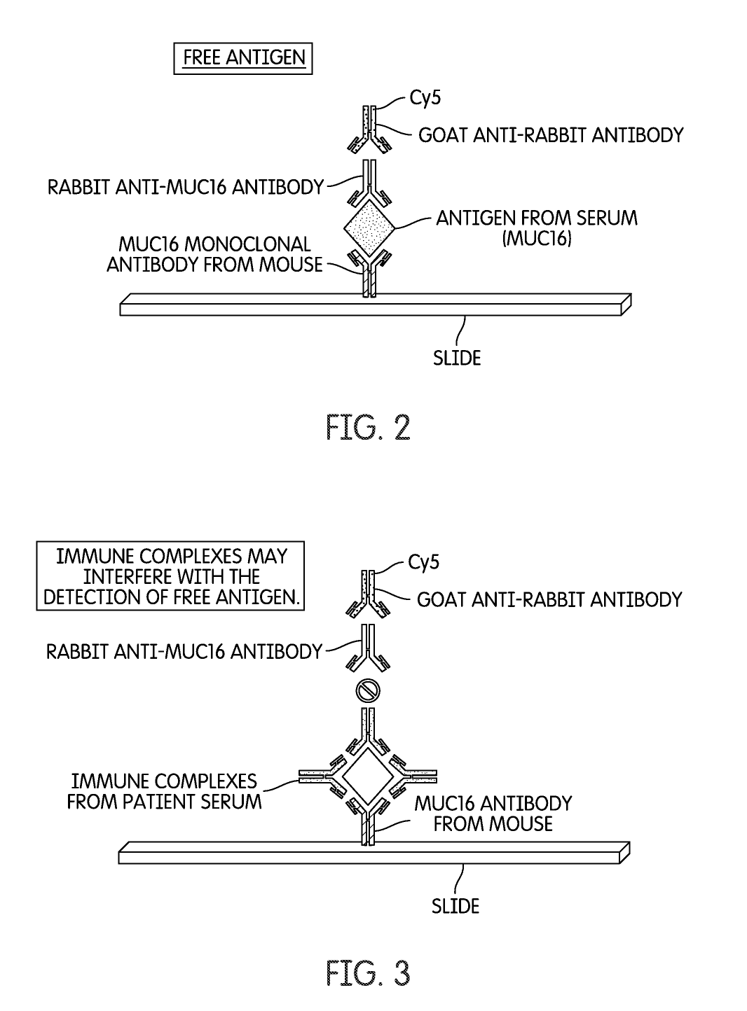

The results herein suggest that some women who present with ovarian cancer and low CA-125 levels may actually have elevated CA-125 levels which are hidden from conventional assays due to the presence of antigen-autoantibody or immune complexes. If so, the detection of CA-125 immune complexes may complement the utility of detecting free CA-125 as a biomarker. A schematic of our hypothesis is shown in FIGS. 2 and 3.

TABLE 1
Fluorescence Levels of CA-125 (MUC16) Antigens and Complexes

| Free Antigen | | | | Immune Complexes | | | |
|---|---|---|---|---|---|---|---|
| MUC16 | | | | MUC16 | | | |
| Free | High | Low | % change | Immune | High | Low | % change |
| Antigen | (CV) | (CV) | (high-low) | Complex | (CV) | (CV) | (high-low) |
| 400 μg | 11429 | 3859 | −66.2 | 400 μg | 2243 | 4807 | 114 |
| | (0.03) | (0.07) | | | (0.03) | (0.08) | |
| 200 μg | 10076 | 3859 | −74.8 | 200 μg | 1662 | 3627 | 118 |
| | (0.07) | (0.15) | | | (0.03) | (0.23) | |
| 100 μg | 8396 | 2862 | −65.9 | 100 μg | 969 | 1944 | 101 |
| | (0.08) | (0.23) | | | (0.01) | (0.12) | |
| Cy3 BSA | 65535 | 65535 | 0 | Cy3 BSA | 65535 | 65535 | 0 |
| | (0.03) | (0.05) | | | (0.03) | (0.01) | |

* The sample labeled “high” has a CA-125 clinically-measured value of 3,331 U/ml and the sample labeled “low” has a CA-125 clinically-measured value of 76.7 U/ml.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1. A method of assaying the amount of CA-125 immune complex in a test biological sample derived from a woman, comprising contacting said test biological sample with an antibody that binds to human anti-CA125 antibody.

2. The method of claim 1, wherein said biological sample is blood, plasma, or serum.

3. The method of claim 2, wherein said human antibody is a human IgG.

4. The method of claim 1, wherein said woman has clinical factors indicating that she is at increased risk of having or developing ovarian cancer or has been previously diagnosed as having ovarian cancer and said method is being used to test for disease recurrence.

5. (canceled)

6. The method of claim 1, wherein said woman has, within the previous three months, had a diagnostic assay performed measuring free CA-125 levels in a sample of blood serum or plasma from said woman.

7. The method of claim 1, wherein said method comprises:
a) obtaining said test biological sample from said woman;
b) incubating said test biological sample with a monoclonal antibody to CA-125, wherein said monoclonal antibody has been immobilized on a solid support and said incubation is carried out under conditions permitting the binding of said monoclonal antibody to said CA-125 when said CA-125 is in an immune complex;
c) at the end of the incubation of step b), separating said solid support with immobilized monoclonal antibody and bound immune complexes from said test biological sample;
d) incubating said solid support with immobilized monoclonal antibody and bound immune complexes obtained in step c) with a detectably labeled antibody that binds to human antibody;
e) at the end of the incubation of step d), separating the solid support with bound detectably labeled antibody from unbound detectably labeled antibody;
f) quantitating the amount of detectable label bound to said solid support.

8. The method of claim 7, wherein said test biological sample is blood, plasma, or serum.

9-12. (canceled)

13. The method of claim 8, wherein said woman has clinical factors indicating that she is at increased risk of having or developing ovarian cancer or has been previously diagnosed as having ovarian cancer and said method is being used to test for disease recurrence.

14. (canceled)

15. The method of claim 8, wherein said woman has, within the previous three months, had a diagnostic assay performed measuring free CA-125 levels in a sample of blood serum or plasma from said woman.

16. A method of diagnostically testing a woman for ovarian cancer, comprising determining the amount of CA-125 immune complexes present in a biological sample obtained from said woman.

17. The method of claim 16, further comprising determining the amount of free CA-125 present in a biological sample obtained from said woman.

18. The method of claim 16, wherein said biological sample is blood, plasma, or serum.

19. The method of claim 18, wherein said human antibody is a human IgG.

20. The method of claim 18, wherein said woman has clinical factors indicating that she is at increased risk of having or developing ovarian cancer.

21. The method of claim 18, wherein said woman has been previously diagnosed as having ovarian cancer and said method is being used to test for disease recurrence.

22. The method of claim 18, wherein said woman has, within the previous three months, had a diagnostic assay performed measuring free CA-125 levels in a sample of blood serum or plasma from said woman.

23. (canceled)

24. The method of claim 17, wherein the amount of free CA-125 in said biological sample is determined by a method comprising:
a) incubating said test biological sample with a monoclonal antibody to CA-125, wherein said monoclonal antibody has been immobilized on a solid support and said incubation is carried out under conditions permitting the binding of said monoclonal antibody to said CA-125;
b) at the end of the incubation of step a), separating said solid support with immobilized monoclonal antibody and bound CA-125 from said test biological sample;
c) incubating said solid support with immobilized monoclonal antibody and bound CA-125 obtained in step b) with a second antibody that binds to CA-125;
d) at the end of the incubation of step c), determining the amount of second antibody bound to the solid support; and wherein, the total amount of free and complexed antibody is compared with CA-125 levels in one or more control samples and the presence of ovarian cancer is indicated by the total amount of CA-125 in said test sample being higher than in said one or more control samples.

25. The method of claim 24, wherein, in step c, said second antibody is unlabeled and:
i) at the end of the incubation of step c) the solid support with bound second antibody is separated from unbound second antibody;
ii) incubating the solid support that has been separated from unbound second antibody with a third antibody that is detectably labeled and that binds to said second antibody;
iii) separating the solid support with bound detectably labeled antibody from unbound detectably labeled antibody; and
iv) quantitating the amount of detectable label bound to said solid support.

26-28. (canceled)

29. A kit comprising a solid support comprising an immobilized monoclonal antibody that binds to CA-125 and one or components selected from the group consisting of: labeled or unlabeled unbound antibody against CA-125; labeled or unlabeled antibody recognizing human antibody; and one or more control samples.

30. The solid support of claim 29, wherein said solid support is a plate or slide made of glass, plastic or metal.
